(19) United States
(12) Patent Application Publication
(10) Pub. No.: US 2008/0281348 A1
(43) Pub. Date: Nov. 13, 2008

(54) MEDICAL DEVICES

(75) Inventor: Boris Warnack, Mountain View, CA (US)

Correspondence Address: WORKMAN NYDEGGER, 1000 EAGLE GATE TOWER,, 60 EAST SOUTH TEMPLE, SALT LAKE CITY, UT, 84111, US

(73) Assignee: ABBOTT LABORATORIES, Abbott Park, IL (US)

(21) Appl. No.: 11/745,844

(22) Filed: May 8, 2007

(51) Int. Cl. A61M 29/02

(52) U.S. Cl. 606/194

(57) ABSTRACT

At least a portion of the medical device may be guided to a desired position within a patient. For example, the medical device may include an expandable member, such as a balloon, which may be guided to a desired position within a patient. When in the desired position, the expandable member may be expanded. Afterwards, the expandable member may be collapsed, and the medical device may be removed from the patient. The expandable member may include one or more cavities, which may facilitate more consistent, predictable and/or compact collapsing of the expandable member.

### BACKGROUND OF THE INVENTION

1. Field of the Invention

The present invention generally relates to medical devices.

2. Description of Related Art

Coronary artery disease occurs when the coronary arteries become narrowed or blocked by a buildup of substances called “plaque.” This plaque buildup may lead to poor blood flow to the heart, which may cause chest pain. If not treated, coronary artery disease may result in a heart attack and, in many cases, death. In fact, coronary artery disease is a leading cause of death in the United States.

Angioplasty is a conventional procedure for treating coronary artery disease. In a typical angioplasty procedure, a medical device is inserted through a blood vessel, such as an artery in the patient's arm, groin or wrist. After insertion, the medical device is guided to a desired position. In particular, the medical device, such as a balloon catheter, typically includes a long flexible tube and an expandable member, such as a balloon, and the medical device may be guided through various blood vessels to a position in which the balloon is located within or near a narrowed portion of an artery. When in the desired position, the balloon may then be inflated to help compress the plaque deposits and/or widen the artery, which may help provide improved blood flow when the balloon is deflated and the medical device is removed. Unfortunately, the balloon may unpredictably deflate into an awkward or undesirable configuration.

If desired, a small tubular device called a “stent” may be placed at or near the compressed plaque deposits and/or the widened artery. For example, in some instances, a balloon catheter may carry a stent to a desired position. When the stent is in the desired position, the balloon may then be inflated to help expand the stent. For example, at least a portion of the balloon may be positioned within the stent's passageway and the inflation may exert a force against the stent's inner wall to expand the stent. Expanding the stent may help compress the plaque deposits and/or widen the artery. Desirably, the stent may remain to help prop the artery open, which may help maintain this improved blood flow and thus increase the success rate of the angioplasty procedures.

### BRIEF SUMMARY OF EMBODIMENTS OF THE INVENTION

A need therefore exists for medical devices that eliminate or diminish the above-described and/or other disadvantages and problems.

One aspect is a medical system that may include a first medical device and a second medical device. The second medical device may include a stent, and the first medical device may be used to guide the stent to a desired position within a patient, such as within a lumen of a coronary artery, other arteries, other blood vessels, other tubular bodily structures, and the like. The second medical device, however, may be used to guide the stent to other positions within any other bodily structure having other characteristics. The first medical device may also be used to help implant the stent at the desired location. For example, the first medical device may include an expandable member, such as a balloon, which may be expanded to help implant the stent.

Another aspect is a medical device and at least a portion of the medical device may be guided to a desired position within a patient. For example, the medical device may include an expandable member, which may be guided to a desired position within a patient, such as within a lumen of a coronary artery, other arteries, other blood vessels, other tubular bodily structures, and the like. The expandable member, however, may be positioned in any other bodily structure having other characteristics. When in the desired position, the expandable member may be expanded. Afterwards, the expandable member may be collapsed, and the medical device may be removed from the patient. The expandable member may be sized and configured for use in an angioplasty procedure and/or for other suitable medical procedures or purposes.

Yet another aspect is a medical device that may include an expandable member that may be sized and configured to expand and collapse. The expandable member may include one or more cavities, which may facilitate more consistent, predictable and/or compact collapsing of the expandable member. For example, the expandable member may include an outer wall, which may include one or more such cavities. The cavities may extend along at least a substantial portion of the length of the expandable member. The cavities may be at least substantially equally spaced apart from each other. The cavities may include one or more pairs of generally opposing cavities. The cavities can have a generally oblong cross-sectional shape, but the cavities may have other suitable cross-sectional shapes. The expandable member may include a balloon, such as a dilation balloon.

Still another aspect is a preform from which an expandable member may be constructed. The preform may include one or more cavities. For example, the preform may include an outer wall, which may include one or more cavities. The cavities may extend along at least a substantial portion of the length of the preform. The cavities may be at least substantially equally spaced apart from each other. The cavities may include one or more pairs of generally opposing cavities. The cavities can have a generally circular cross-sectional shape, but the cavities may have other suitable cross-sectional shapes.

Another aspect is a method for constructing an expandable member of a medical device. The method may include creating a preform and creating an expandable member from the preform. The preform can be created using an extrusion molding process. The preform can be formed into a parison that may be blow molded into an expandable member. The preform may be created using other molding and/or manufacturing processes and the expandable member may be created from the preform using other molding and/or manufacturing processes.

For purposes of summarizing, some aspects, advantages and features of some embodiments of the invention have been described in this summary. Not necessarily all of (or any of) these summarized aspects, advantages or features will be embodied in any particular embodiment of the invention. Some of these summarized aspects, advantages and features and other aspects, advantages and features may become more fully apparent from the following detailed description and the appended claims.

### BRIEF DESCRIPTION OF THE DRAWINGS

The appended drawings contain figures of preferred embodiments to further illustrate and clarify the above and other aspects, advantages and features of the present invention. It will be appreciated that these drawings depict only preferred embodiments of the invention and are not intended to limit its scope. The invention will be described and explained with additional specificity and detail through the use of the accompanying drawings in which:

FIG. 1 is a diagram illustrating an exemplary medical system including an exemplary medical device, illustrating an exemplary expandable member;

FIG. 2 is a diagram of a portion of the medical device shown in FIG. 1 with another exemplary medical device;

FIG. 3 is an exploded view of a portion of the medical devices shown in FIG. 2;

FIG. 4 is a cross-sectional view of an exemplary configuration of the expandable member shown in FIG. 1;

FIG. 5 is a cross-sectional view of an exemplary configuration of the expandable member shown in FIG. 1;

FIG. 6 is a cross-sectional view of an exemplary configuration of the expandable member shown in FIG. 1;

FIG. 7 is a cross-sectional view of an exemplary configuration of the expandable member shown in FIG. 1;

FIG. 8 is a cross-sectional view of an exemplary configuration of the expandable member shown in FIG. 1;

FIG. 9 is an enlarged cross-sectional view of a portion of an exemplary configuration of the expandable member shown in FIG. 1;

FIG. 10 is a flowchart illustrating an exemplary method;

FIG. 11 is a cross-sectional view of an exemplary preform, which may be used to form the expandable member shown in FIG. 1;

FIG. 12 is a cross-sectional view of an exemplary preform, which may be used to form the expandable member shown in FIG. 1;

FIG. 13 is a cross-sectional view of an exemplary preform, which may be used to form the expandable member shown in FIG. 1;

FIG. 14 is a cross-sectional view of an exemplary preform, which may be used to form the expandable member shown in FIG. 1;

FIG. 15 is a cross-sectional view of an exemplary preform, which may be used to form the expandable member shown in FIG. 1; and

FIG. 16 is an enlarged cross-sectional view of a portion of an exemplary preform, which may be used to form the expandable member shown in FIG. 1

### DETAILED DESCRIPTION OF THE PREFERRED EMBODIMENTS

The present invention is generally directed towards a medical device. The principles of the present invention, however, are not limited to medical devices. It will be understood that, in light of the present disclosure, the medical device disclosed herein can be successfully used in connection with other types of devices. A detailed description of the medical device now follows.

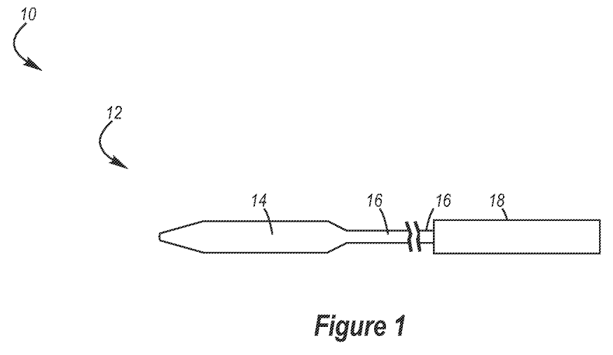

As shown in FIG. 1, a medical system 10 may include one or more medical devices, such as a medical device 12. The medical device 12 may include an expandable member 14, such as a balloon. The medical device 12 may also include a catheter 16, a handle 18, a guide wire (not shown) and/or other suitable components. The medical device 12, however, does not require any of these particular components. For example, the medical device 12 does not require the handle 18 and may include, for example, a port through which the medical device 12 and/or the catheter 16 may receive a liquid, a gas and/or other suitable substance to help expand the expandable member 14.

At least a portion of the medical device 12 may be guided to a desired position within a patient, such as within a lumen of a coronary artery, other arteries, other blood vessels, other tubular or non-tubular bodily structures, and the like. For example, at least a portion of the medical device 12 may be guided through various blood vessels to a position in which the expandable member 14 is located within or near a narrowed portion of an artery. It will be appreciated, however, that the expandable member 14 may be positioned in any other bodily structure having other characteristics.

When in the desired position, the expandable member 14 may be expanded. Afterwards, the expandable member 14 may be collapsed and the medical device 12 may be removed from the patient. For example, the expandable member 14 may include a balloon, such as a dilation balloon. The balloon may be inflated to help compress plaque deposits and/or widen an artery, which may help provide improved blood flow when the balloon is deflated and the medical device 12 is removed. Thus, in some embodiments, the medical device 12 may be used for angioplasty, such as percutaneous transluminal angioplasty and/or percutaneous transluminal coronary angioplasty. It will be appreciated, however, that the medical device 12 need not be used for angioplasty and may be used for other suitable medical procedures or purposes.

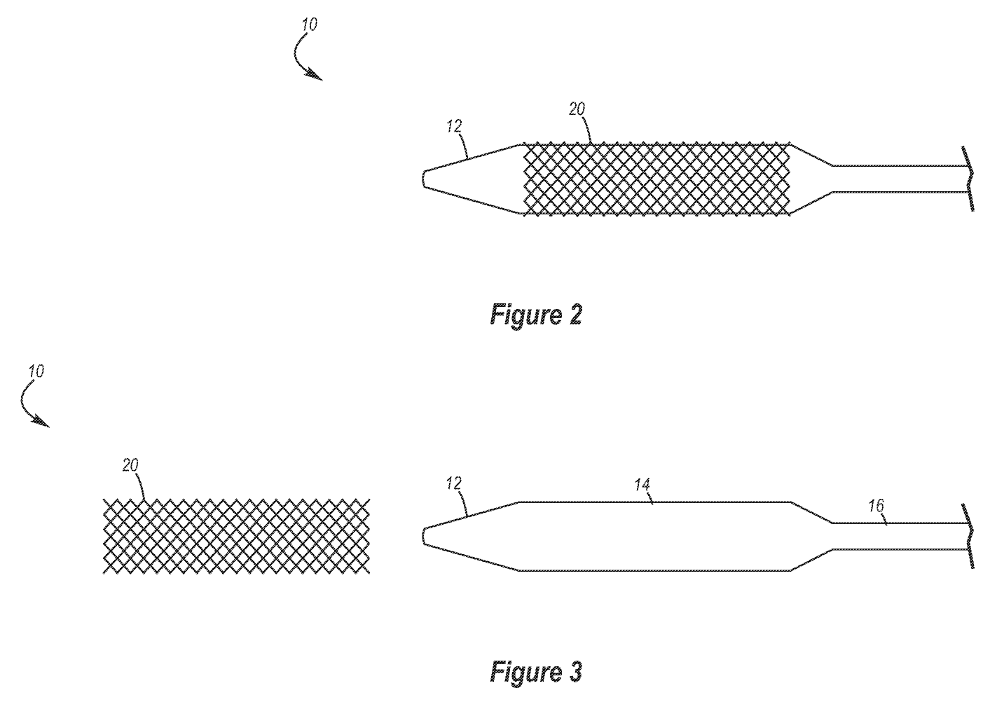

As shown in FIGS. 2-3, the medical system 10 may also include another medical device, such as a stent 20. The stent 20 can be constructed from metal mesh (such as steel mesh) formed into a tubular configuration having a cross section that is generally circular, square, rectangular, oval, oblong and/or other suitable shape. It will be appreciated, however, that the stent 20 may have a variety of other suitable constructions, configurations and/or shapes depending, for example, upon the particular stent. It will also be appreciated that the stent 20 need not be constructed from metal and may be constructed from other suitable materials.

At least a portion of the medical device 12 may be used to guide the stent 20 to a desired position within a patient, such as within a lumen of a coronary artery, other arteries, other blood vessels, other tubular or non-tubular bodily structures, and the like. For example, as shown in FIG. 2, the stent 20 may be connected to the medical device and the stent 20 and at least a portion of the medical device 12 may be guided through various blood vessels to a position in which the stent 20 is located within or near a narrowed portion of an artery.

When in the desired position, the stent 20 may be implanted, and the medical device 12 may be removed from the patient. The stent 20 can be expanded to implant the stent at the desired position. For instance, in one embodiment, the medical device 12 may include an expandable member 14, such as a balloon, that may be expanded to help expand the stent 20. In particular, at least a portion of the balloon may be positioned within the stent's passageway and may be inflated to exert a force against the stent's inner wall to expand the stent. Expanding the stent 20 may help compress plaque deposits and/or widen an artery. Desirably, the stent 20 may remain to help prop the artery open, which may help provide improved blood flow when the balloon is deflated and the medical device 12 is removed. Thus, in some embodiments, the medical device 12 may be used for stent placement. It will be appreciated, however, that the stent 20 need not be balloon-expandable and may be self-expandable. It will also be appreciated that the stent 20 may be implanted using a variety of other suitable methods and/or at a variety of other suitable locations within a patient.

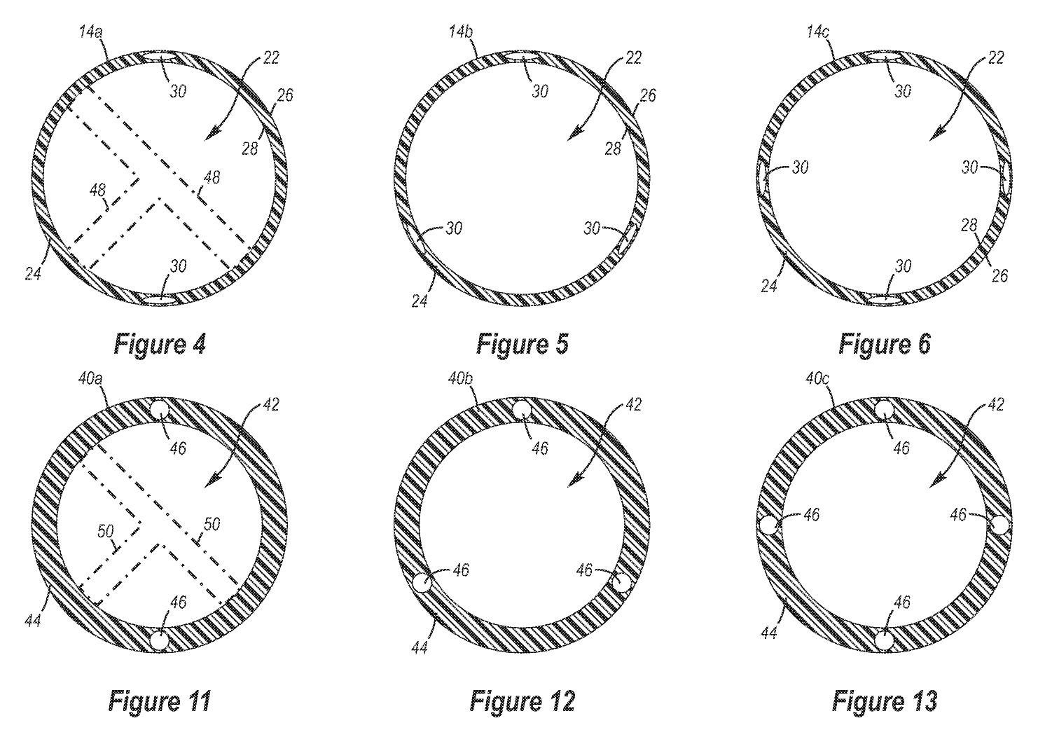

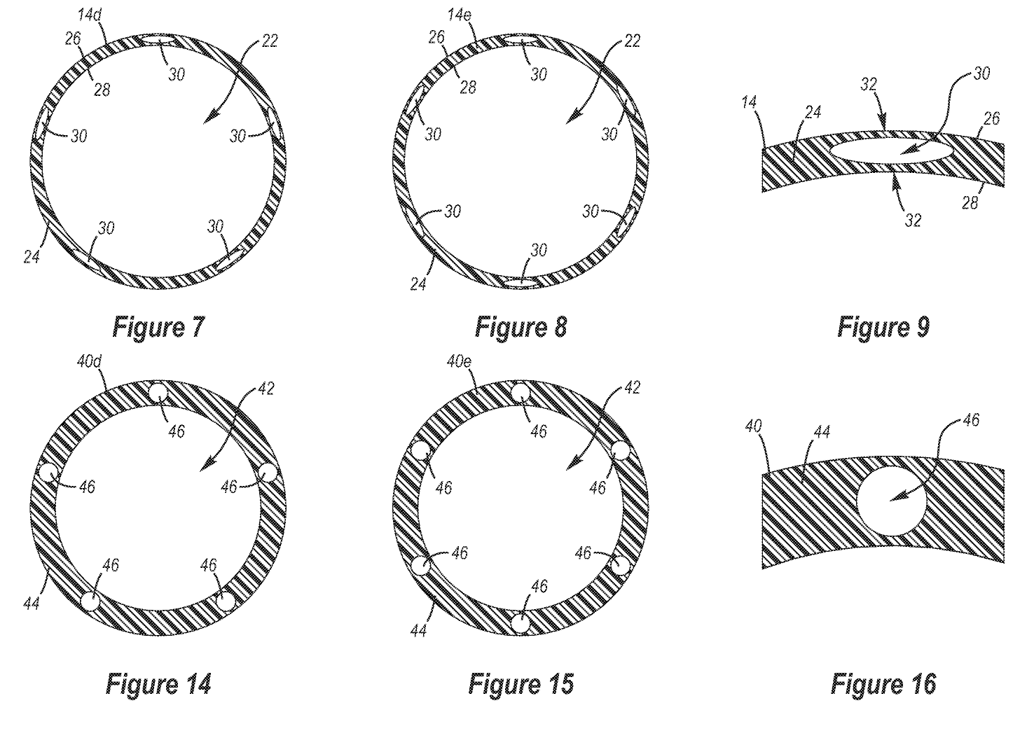

The expandable member 14 may have a tubular shape. In particular, as shown in FIGS. 4-8, the expandable member 14 includes at least one cavity 22, and the expandable member 14 and/or the cavity 22 may have a generally circular cross-sectional shape. It will be appreciated, however, that the cavity 22 and/or the tubular expandable member 14 may have a generally circular, square, rectangular, oval, oblong and/or other suitable cross-sectional shape. It will also be appreciated that the expandable member 14 need not have a tubular shape and that the expandable member 14 may have a variety of other suitable shapes and/or configurations.

The cavity 22 can be sized and configured to receive a liquid, a gas and/or other suitable substance to help expand the expandable member 14. In further detail, the expandable member 14 can include an outer wall 24 having an outer surface 26 and an inner surface 28. The inner surface 28 of the wall 24 can define at least a portion of the cavity 22. Consequently, a liquid, gas and/or other suitable substance may, as it enters the cavity 22, help exert increased pressure against the inner surface 28 of the wall 24, which may cause at least a portion of the expandable member 14, such as the wall 24, to expand. As a liquid, gas and/or other suitable substance exits the cavity 22, this pressure may decrease, which may cause the wall 24 and the expandable member 14 to collapse. As discussed in further detail below, the wall 24 may also include one or more cavities 30 sized and configured to facilitate more consistent, predictable and/or compact collapsing of the expandable member 14 and the wall 24.

As shown in FIGS. 4-9, the wall 24 of an expandable member 14 may include one or more cavities 30. The cavities 30 are generally disposed between the outer and inner surfaces 26, 28 of the wall 24, and the cavities 30 may help define a pair of spaced apart wall portions 32 as shown in FIG. 9. As the wall 24 and the expandable member 14 collapse, the wall 24 and the expandable member 14 advantageously tend to fold proximate the cavities 30 and the wall portions 32, facilitating more consistent, predictable and/or compact collapsing.

The cavities 30 and/or the wall portions 32 can extend along at least a substantial portion of the length of the expandable member 14, such as at least one-tenth, one-fifth, one-fourth, one-third or one-half of the expandable member's length. Desirably, when the cavities 30 and the wall portions 32 extend along at least a substantial portion of the length of the expandable member 14, the cavities 30 and the wall portions 32 may help facilitate more consistent, predictable and/or compact collapsing of the expandable member 14 and the wall 24. It will be appreciated, however, the cavities 30 and/or the wall portions 32 may be longer or shorter and may even extend along the entire length of the expandable member 14. As such, the cavities 30 can extend from a proximal end of the expandable member 14 toward the distal end of the expandable member 14, or vice versa.

The wall 24 of the expandable member 14 may include any number of cavities 30. For example, as shown in FIGS. 4-8, the walls 24 of the expandable members 14a,14b, 14c, 14d, 14e may respectively include two, three, four, five or six cavities 30—which may help form corresponding folds in the expandable member 14 and/or the wall 24 as they collapse. Of course, a wall 24 may include more or fewer cavities 30 and may even include a single cavity 30, if desired. It will be appreciated, however, that a wall 24 does not require any cavities 30.

The wall 24 of the expandable member 14 may include any suitable arrangement of cavities 30. In some embodiments, the wall 24 may include a plurality of cavities 30 that are at least substantially equally spaced apart from each other. For example, as shown in FIGS. 4-8, the walls 24 of the expandable members 14a, 14b, 14c, 14d, 14e may include two, three, four, five or six equally (or at least substantially equally) spaced apart cavities 30, respectively. Also, in some embodiments, the wall 24 may include at least one pair of generally opposing cavities 30. For example, the walls 24 of the expandable members 14a, 14c, 14e may include one, two or three pairs of generally opposing cavities, respectively. Desirably, when a wall 24 includes cavities 30 that are at least substantially equally spaced apart from each other and/or when the wall 24 includes at least one pair of generally opposing cavities 30, the cavities 30 may help facilitate more consistent, predictable and/or compact collapsing of the expandable member 14 and the wall 24. It will be appreciated, however, that the wall 24 of the expandable member 14 do not require cavities 30 that are at least substantially equally spaced apart from each other or any pairs of generally opposing cavities 30 and that the cavities may be relatively positioned in any other desired arrangement.

The cavities 30 can have a generally oblong cross-sectional shape, which may help facilitate more consistent, predictable and/or compact collapsing of the expandable member 14 and the wall 24. The generally oblong cross-sectional shape may have a minor axis that is oriented in a generally perpendicular orientation relative to the outer surface 26 and/or the inner surface 28 of the wall 24. The generally oblong cross-sectional shape may have a major axis that is generally aligned with the outer surface 26 and/or the inner surface 28 of the wall 24. The cavities 30, however, do not require a generally oblong cross-sectional shape and may have a generally circular, square, rectangular, oval, oblong and/or other suitable cross-sectional shape positioned in any suitable orientation. In addition, some or all of the cavities 30 may have the same cross-sectional shape or different cross-sectional shapes depending, for example, upon the particular configuration of the expandable member 14.

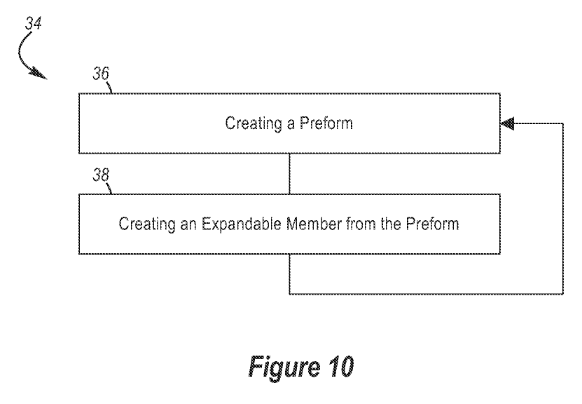

As shown in FIG. 10, a method 34 may be used to help construct at least a portion of a medical device, such as an expandable member 14. In particular, a preform may be created at a block 36, and an expandable member 14 may be created from the preform at a block 38. For example, the expandable members 14a, 14b, 14c, 14d, 14e in FIGS. 4-8 may be respectively created from preforms 40a, 40b, 40c, 40d, 40e in FIGS. 11-15. It will be appreciated, however, that other expandable members 14 having other suitable configurations may be created from the preforms 40a, 40b, 40c, 40d, 40e.

In some embodiments, the preform 40 may be created using an extrusion molding process, as represented by block 36. The extrusion-molded preform 40 may be formed into a parison that, as represented by block 38, may be blow molded into an expandable member 14. In particular, the parison may be stretch-blow molded into an expandable member 14. It will be appreciated, however, the preform 40 may be created using other molding and/or manufacturing processes and that the expandable member 14 may be created from the preform 40 using other molding and/or manufacturing processes.

The preform 40 may have a tubular shape. In particular, as shown in FIGS. 11-15, the preform 40 can include at least one cavity 42, and the preform 40 and/or the cavity 42 may have a generally circular cross-sectional shape. It will be appreciated, however, that the cavity 42 and/or the tubular preform 40 may have a generally circular, square, rectangular, oval, oblong and/or other suitable cross-sectional shape. It will also be appreciated that the preform 40 need not have a tubular shape and that the preform 40 may have a variety of other suitable shapes and/or configurations.

As shown in FIGS. 11-15, the preform 40 can include an outer wall 44. The wall 44 may include one or more cavities 46. The cavities 46 are generally disposed between the outer and inner surfaces of the wall 44. In some embodiments, the wall 44 and the cavities 46 of the preform 40 may be respectively formed into the wall 24 and the cavities 30 of the expandable member 14, for example, as represented by block 38 in FIG. 10.

The cavities 46 can extend along at least a substantial portion of the length of the preform 40, such as at least one-tenth, one-fifth, one-fourth, one-third or one-half of the preform's length. It will be appreciated, however, that the cavities 46 may be longer or shorter and may even extend along the entire length of the preform 40. As such, the cavities 46 can extend from a proximal end of the preform 40 toward the distal end of the preform 40, or vice versa.

The wall 44 of the preform 40 may include any number of cavities 46. For example, as shown in FIGS. 11-15, the walls 44 of the preforms 40a, 40b, 40c, 40d, 40e may respectively include two, three, four, five or six cavities 46. Of course, a wall 44 may include more or fewer cavities 46 and may even include a single cavity 46, if desired. It will be appreciated, however, that a wall 44 does not require any cavities 46.

The wall 44 of the preform 40 may include any suitable arrangement of cavities 46. In some embodiments, the wall 44 may include a plurality of cavities 46 that are at least substantially equally spaced apart from each other. For example, as shown in FIGS. 11-15, the walls 44 of the preforms 40a, 40b, 40c, 40d, 40e may include two, three, four, five or six equally (or at least substantially equally) spaced apart cavities 46, respectively. Also, in some embodiments, the wall 44 may include at least one pair of generally opposing cavities 46. For example, the walls 44 of the preforms 40a, 40c, 40e may include one, two or three pairs of generally opposing cavities, respectively. It will be appreciated, however, that the wall 44 of the preform 40 do not require cavities 46 that are at least substantially equally spaced apart from each other or any pairs of generally opposing cavities 46 and that the cavities may be relatively positioned in any other desired arrangement.

The cavities 46 can have a generally circular cross-sectional shape. The cavities 46, however, do not require a generally circular cross-sectional shape and may have a generally circular, square, rectangular, oval, oblong and/or other suitable cross-sectional shape positioned in any suitable orientation. In addition, some or all of the cavities 46 may have the same cross-sectional shape or different cross-sectional shapes depending, for example, upon the particular configuration of the preform 40.

As shown in FIGS. 4 and 11, the expandable member 14 and the preform 40 may include one or more interior walls 48, 50. The interior walls 48, 50 may, for example, be sized and configured to allow portions of the expandable member 14 to expand, while allowing other portions to remain generally the same size and/or to carry bodily fluids, house guide wires, and/or perform other suitable functions. The expandable member 14 and the preform 40, however, do not require any interior walls 48, 50.

In one or more embodiments, a preform for constructing at least a portion of a balloon of a medical device may be provided. The preform may include an outer wall including an inner surface and an outer surface; and a plurality of cavities formed in the wall generally between the inner and outer surfaces of the wall, the plurality of cavities being at least substantially equally spaced apart from each other. The plurality of cavities may include three or more cavities that are at least substantially equally spaced apart from each other. The plurality of cavities may include four or more cavities that are at least substantially equally spaced apart from each other. The balloon may include a dilation balloon. At least one of the plurality of cavities may have a generally circular cross-sectional shape.

In one or more embodiments, a preform for constructing at least a portion of a balloon of a medical device may be provided. The preform may include an outer wall including an inner surface and an outer surface; and a plurality of cavities formed in the wall generally between the inner and outer surfaces of the wall; and the plurality of cavities may include at least one pair of generally opposing cavities. The at least one pair of generally opposing cavities may include at least two pairs of generally opposing cavities. The balloon may include a dilation balloon. At least one of the plurality of cavities may have a generally circular cross-sectional shape.

In one or more embodiments, a method for constructing a balloon of a medical device may be provided. The method may include creating a perform; and creating a balloon from the perform. The preform may include an outer wall including an inner surface and an outer surface; and at least one cavity formed in the wall generally between the inner and outer surfaces of the wall, the at least one cavity extending along at least a substantial portion of the length of the perform. Creating a preform may include extrusion molding the preform. Creating a balloon from the perform may include forming the preform into a parison; and blow molding the parison into a balloon. Creating a balloon from the perform may include forming the preform into a parison; and stretch-blow molding the parison into a balloon. Creating a balloon from the perform may include forming the preform into a parison; and blow molding the parison into a balloon. Creating a balloon from the perform may include forming the preform into a parison; and stretch-blow molding the parison into a balloon. The balloon may be sized and configured to be expanded and collapsed within a blood vessel; and the balloon may include an expandable and collapsible outer wall including an inner surface and an outer surface; and at least one cavity formed in the expandable and collapsible outer wall generally between the inner and outer surfaces of the expandable and collapsible outer wall, the at least one cavity formed in the expandable and collapsible outer wall extending along at least a substantial portion of the length of the balloon.

In one or more embodiments, a method for constructing a balloon of a medical device may be provided. The method may include creating a perform; and creating a balloon from the perform. The preform may include an outer wall including an inner surface and an outer surface; and a plurality of cavities formed in the wall generally between the inner and outer surfaces of the wall, the plurality of cavities being at least substantially equally spaced apart from each other. Creating a preform may include extrusion molding the preform. Creating a balloon from the perform may include forming the preform into a parison; and blow molding the parison into a balloon. Creating a balloon from the perform may include forming the preform into a parison; and stretch-blow molding the parison into a balloon. Creating a balloon from the perform may include forming the preform into a parison; and blow molding the parison into a balloon. Creating a balloon from the perform may include forming the preform into a parison; and stretch-blow molding the parison into a balloon. The balloon may sized and configured to be expanded and collapsed within a blood vessel; and the balloon may include an expandable and collapsible outer wall including an inner surface and an outer surface; and a plurality of cavities formed in the expandable and collapsible outer wall generally between the inner and outer surfaces of the expandable and collapsible outer wall, the plurality of cavities formed in the expandable and collapsible outer wall being at least substantially equally spaced apart from each other.

In one or more embodiments, a method for constructing a balloon of a medical device may be provided. The method may include creating a perform; and creating a balloon from the perform. The preform may include an outer wall including an inner surface and an outer surface; and a plurality of cavities formed in the wall generally between the inner and outer surfaces of the wall, the plurality of cavities may include at least one pair of generally opposing cavities. Creating a preform may include extrusion molding the preform. Creating a balloon from the perform may include forming the preform into a parison; and blow molding the parison into a balloon. Creating a balloon from the perform may include forming the preform into a parison; and stretch-blow molding the parison into a balloon. Creating a balloon from the perform may include forming the preform into a parison; and blow molding the parison into a balloon. Creating a balloon from the perform may include forming the preform into a parison; and stretch-blow molding the parison into a balloon. The balloon may be sized and configured to be expanded and collapsed within a blood vessel; and the balloon may include an expandable and collapsible outer wall including an inner surface and an outer surface; and a plurality of cavities formed in the expandable and collapsible outer wall generally between the inner and outer surfaces of the expandable and collapsible outer wall, the plurality of cavities formed in the expandable and collapsible outer wall may include at least one pair of generally opposing cavities.

In one or more embodiments, a method for using a medical device may be provided. The method may include placing an expandable member of a medical device at a location within a patient's body. The expandable member may include an outer wall including an inner surface and an outer surface; and at least one cavity formed in the wall generally between the inner and outer surfaces of the wall, the at least one cavity extending along at least a substantial portion of the length of the expandable member. The method may also include expanding the outer wall; and collapsing the outer wall. The method may also include removing the expandable member from the patient's body. The at least one cavity may include a plurality of cavities. The at least one cavity may include a plurality of cavities that are at least substantially equally spaced apart from each other. The at least one cavity may include three or more cavities that are at least substantially equally spaced apart from each other. The at least one cavity may include four or more cavities that are at least substantially equally spaced apart from each other. The at least one cavity may include at least one pair of generally opposing cavities. The at least one cavity may include at least two pairs of generally opposing cavities. The expandable member may include a balloon. The expandable member may include a dilation balloon. The at least one cavity may have a generally oblong cross-sectional shape. The location within a patient's body may include a location within a blood vessel.

In one or more embodiments, a method for using a medical device may be provided. The method may include placing a balloon of a medical device at a location within a patient's body, and the balloon may include an outer wall including an inner surface and an outer surface; and a plurality of cavities formed in the wall generally between the inner and outer surfaces of the wall, the plurality of cavities being at least substantially equally spaced apart from each other. The method may also include expanding the outer wall; and collapsing the outer wall. The method may also include removing the balloon from the patient's body. At least two of the plurality of cavities may extend along at least a substantial portion of the length of the balloon. The plurality of cavities may include three or more cavities that are at least substantially equally spaced apart from each other. The plurality of cavities may include four or more cavities that are at least substantially equally spaced apart from each other. The balloon may include a dilation balloon. At least one of the plurality of cavities may have a generally oblong cross-sectional shape. The location within a patient's body may include a location within a blood vessel.

In one or more embodiments, a method for using a medical device may be provided. The method may include placing a balloon of a medical device at a location within a patient's body, and the balloon may include an outer wall including an inner surface and an outer surface; and a plurality of cavities formed in the wall generally between the inner and outer surfaces of the wall. The plurality of cavities may include at least one pair of generally opposing cavities. The method may also include expanding the outer wall; and collapsing the outer wall. The method may also include removing the balloon from the patient's body. At least two of the plurality of cavities may extend along at least a substantial portion of the length of the balloon. The at least one pair of generally opposing cavities may include at least two pairs of generally opposing cavities. The balloon may include a dilation balloon. The at least one of the plurality of cavities may have a generally oblong cross-sectional shape. The location within a patient's body may include a location within a blood vessel.

The methods, systems and devices described above require no particular component, function or feature. Thus, any described component, function or feature—despite its advantages—is optional. Also, some or all of the described components, functions and features described above may be used in connection with any number of other suitable components, functions and features.

Although this invention has been described in terms of certain preferred embodiments, other embodiments apparent to those of ordinary skill in the art are also within the scope of this invention. Accordingly, the scope of the invention is intended to be defined only by the claims which follow.

What is claimed is:

1. A medical device comprising:
an expandable member sized and configured to be expanded and collapsed within a blood vessel, the expandable member comprising:
an expandable and collapsible outer wall including an inner surface and an outer surface; and
at least one cavity formed in the wall generally between the inner and outer surfaces of the wall, the at least one cavity extending along at least a substantial portion of the length of the expandable member.

2. The medical device as in claim 1, wherein the at least one cavity comprises a plurality of cavities.

3. The medical device as in claim 1, wherein the at least one cavity comprises a plurality of cavities that are at least substantially equally spaced apart from each other.

4. The medical device as in claim 1, wherein the at least one cavity comprises three or more cavities that are at least substantially equally spaced apart from each other.

5. The medical device as in claim 1, wherein the at least one cavity comprises four or more cavities that are at least substantially equally spaced apart from each other.

6. The medical device as in claim 1, wherein the at least one cavity comprises at least one pair of generally opposing cavities.

7. The medical device as in claim 1, wherein the at least one cavity comprises at least two pairs of generally opposing cavities.

8. The medical device as in claim 1, wherein the expandable member comprises a balloon.

9. The medical device as in claim 1, wherein the expandable member comprises a dilation balloon.

10. The medical device as in claim 1, wherein the at least one cavity has a generally oblong cross-sectional shape.

11. A medical device comprising:
a balloon sized and configured to be expanded and collapsed within a blood vessel, the balloon comprising:
an expandable and collapsible outer wall including an inner surface and an outer surface; and
a plurality of cavities formed in the wall generally between the inner and outer surfaces of the wall, the plurality of cavities being at least substantially equally spaced apart from each other.

12. The medical device as in claim 11, wherein at least two of the plurality of cavities extend along at least a substantial portion of the length of the balloon.

13. The medical device as in claim 11, wherein the plurality of cavities comprises three or more cavities that are at least substantially equally spaced apart from each other.

14. The medical device as in claim 11, wherein the plurality of cavities comprises four or more cavities that are at least substantially equally spaced apart from each other.

15. The medical device as in claim 11, wherein the balloon comprises a dilation balloon.

16. The medical device as in claim 11, wherein at least one of the plurality of cavities has a generally oblong cross-sectional shape.

17. A medical device comprising:
a balloon sized and configured to be expanded and collapsed within a blood vessel, the balloon comprising:
an expandable and collapsible outer wall including an inner surface and an outer surface; and
a plurality of cavities formed in the wall generally between the inner and outer surfaces of the wall, the plurality of cavities comprising at least one pair of generally opposing cavities.

18. The medical device as in claim 17, wherein at least two of the plurality of cavities extend along at least a substantial portion of the length of the balloon.

19. The medical device as in claim 17, wherein the at least one pair of generally opposing cavities comprises at least two pairs of generally opposing cavities.

20. The medical device as in claim 17, wherein the balloon comprises a dilation balloon.

21. The medical device as in claim 17, wherein at least one of the plurality of cavities has a generally oblong cross-sectional shape.

22. A preform for constructing at least a portion of a balloon of a medical device, the preform comprising:
an outer wall including an inner surface and an outer surface; and
at least one cavity formed in the wall generally between the inner and outer surfaces of the wall, the at least one cavity extending along at least a substantial portion of the length of the preform.

23. The preform as in claim 22, wherein the at least one cavity comprises a plurality of cavities.

24. The preform as in claim 22, wherein the at least one cavity comprises a plurality of cavities that are at least substantially equally spaced apart from each other.

25. The preform as in claim 22, wherein the at least one cavity comprises three or more cavities that are at least substantially equally spaced apart from each other.

26. The preform as in claim 22, wherein the at least one cavity comprises four or more cavities that are at least substantially equally spaced apart from each other.

27. The preform as in claim 22, wherein the at least one cavity comprises at least one pair of generally opposing cavities.

28. The preform as in claim 22, wherein the at least one cavity comprises at least two pairs of generally opposing cavities.

29. The preform as in claim 22, wherein the balloon comprises a dilation balloon.

30. The preform as in claim 22, wherein the at least one cavity has a generally circular cross-sectional shape.
